(19) United States
(12) Patent Application Publication
(10) Pub. No.: US 2005/0149117 A1
(43) Pub. Date: Jul. 7, 2005

(54) Apparatus and methods for delivering sealing materials during a percutaneous procedure to facilitate hemostasis

(76) Inventors: Farhad Khosravi, San Mateo, CA (US); Celso J. Bagaoisan, Union City, CA (US); Suresh S. Pai, Mountain View, CA (US); Scott R. Sershen, Belmont, CA (US)

Correspondence Address: BINGHAM, MCCUTCHEN LLP, THREE EMBARCADERO CENTER, 18 FLOOR, SAN FRANCISCO, CA, 94111-4067, US

(21) Appl. No.: 10/745,946

(22) Filed: Dec. 24, 2003

(51) Int. Cl. A61B 17/08

(52) U.S. Cl. 606/215

(57) ABSTRACT

Apparatus and methods are provided for sealing a puncture extending through tissue to a blood vessel. A delivery sheath is introduced-into the puncture, and a sealing compound, e.g., precursor polymers that mix to create a hydrogel, is introduced through the delivery sheath into the puncture. The delivery sheath is then removed, an introducer sheath is advanced through the puncture into the vessel, and instruments are introduced through the introducer sheath into the vessel to perform a medical procedure. After completing the procedure, the introducer sheath is withdrawn. The sealing compound may expand into the puncture and/or tissue recoil of the surrounding tissue may cause the sealing compound to at least partially occlude the puncture to facilitate sealing and/or hemostasis.

### FIELD OF INVENTION

The present invention relates generally to systems and methods for sealing punctures in a body, and, more particularly, to systems and methods for facilitating hemostasis of a vascular puncture extending through tissue into a blood vessel.

### BACKGROUND

Apparatus and methods are known for accessing a patient's vasculature percutaneously for performing a procedure within the vasculature, and for sealing the puncture that results after completing the procedure. For example, a hollow needle may be inserted through a patient's skin and overlying tissue into a blood vessel. A guide wire is-then passed through the needle into the blood vessel, whereupon the needle is removed. An introducer sheath is then advanced over the guide wire into the vessel, e.g., in conjunction with or subsequent to one or more dilators. A catheter or other device may be advanced through the introducer sheath and over the guide wire into a position for performing a medical procedure within the patient's body. In this manner, the introducer sheath facilitates introducing various instruments into the vessel, while minimizing trauma to the vessel wall and blood loss.

Upon completing the procedure, the instrument(s) and introducer sheath are removed, leaving a puncture extending between the skin and the vessel. To seal the puncture, external pressure may be applied to the overlying tissue, e.g., manually and/or using sandbags, until hemostasis occurs. This procedure, however, can be time consuming and expensive, requiring as much as an hour of a medical professional's time. It is also uncomfortable for the patient, and may require the patient to remain immobilized in an operating room, catheter lab, or holding area. In addition, a risk of hematoma exists from bleeding before hemostasis occurs.

Various apparatus and methods have been suggested for sealing a percutaneous puncture instead of or in addition to using external pressure. For example, U.S. Pat. No. 5,108,421 to Fowler discloses using a collagen plug that is delivered into a puncture through tissue. After completing the procedure, the introducer sheath and/or guide wire used to access the patient's vasculature via the puncture are removed. In one embodiment, a catheter is inserted through the puncture into the blood vessel. A balloon on the catheter is expanded and then retracted until the balloon is disposed adjacent the puncture at the wall of the vessel to provide temporary hemostasis between the vessel and the puncture. A plug is then advanced into the puncture until the plug contacts the balloon, thereby preventing the plug from entering the vessel. Once the plug is positioned within the puncture, the balloon is deflated and withdrawn, leaving the plug to expand and seal the puncture and/or promote hemostasis.

By way of another example, U.S. Pat. Nos. 5,192,302 and 5,222,974 issued to Kensey et al. describe using a biodegradable collagen plug that may be delivered through an introducer sheath into a puncture site.

Such sealing methods generally involve introducing plugs or other materials into the puncture after completing the procedure and after removing the introducer sheath. With the introducer sheath removed, there is substantial risk of hematoma within the tissue surrounding the puncture as blood from the vessel leaks into the puncture, which may be uncomfortable and/or harmful to the patient. Further, temporary hemostasis devices for isolating the vessel from the puncture may be difficult to use effectively and/or may be expensive. Despite attempts to isolate the vessel from the puncture while delivering a plug or other sealing material, the sealing material may still leak and/or become exposed in the vessel, where the sealing material risk creating embolic material in the vessel that may harm the patient.

Accordingly, improved systems and methods for sealing punctures, e.g., a percutaneous puncture communicating with a blood vessel, would be useful.

### SUMMARY OF THE INVENTION

The present invention is directed to apparatus, systems, and methods for sealing punctures in a body, and more particularly, to systems and methods for facilitating hemostasis of a vascular puncture extending through tissue into a blood vessel. In various embodiments, the present invention includes systems and methods for delivering a hydrogel or other sealing compound into a percutaneous puncture extending from a patient's skin to a blood vessel or other body lumen before or while performing a vascular procedure to facilitate sealing the puncture after the procedure.

In accordance with one aspect of the invention, a method is provided for sealing a puncture extending through intervening tissue to a body lumen. In particular, before performing and/or completing a medical procedure via the puncture, a sealing compound may be introduced into the puncture, e.g., to “pre-seal” the puncture. In exemplary embodiments, the sealing compound is a hydrogel, e.g., in a liquid, powder, solid, lyophilized, and/or other dehydrated form, synthetic pro-thrombotics, or biological pro-thrombotics, such as thrombin, collagen, or other pro-thrombotic protein-based material and/or bioabsorbable material, e.g., in a solid, paste, or liquid form. In addition or alternatively, the sealing compound may include therapeutic and/or pharmaceutical agents, e.g., to promote healing, prevent infection and/or other adverse medical events. In one embodiment, the sealing compound includes liquid precursor polymer components injected from one or more syringes to create a hydrogel in-situ within the puncture. Thus, as used herein “sealing compound” or “sealant” may include any agent, material, or device that may aid in achieving a substantially fluid-tight barrier and/or hemostasis within an organ or tissue that is at risk of post-surgical leakage.

In an exemplary embodiment, a delivery sheath (e.g., an introducer sheath, catheter, or other tubular member), or other delivery device may be introduced into the puncture, and the delivery sheath may be positioned such that a distal end of the delivery sheath is disposed within the intervening tissue. The sealing compound is then injected through the delivery sheath such that the sealing compound at least partially fills the puncture extra-vascularly and/or permeates into the intervening tissue surrounding the puncture.

For example, the delivery sheath may be advanced over a needle used to create the puncture. The needle is inserted into the patient's skin, through the intervening tissue, and into a blood vessel or other body lumen, e.g., using known methods, followed by insertion of the guide wire. With the needle extending into the body lumen (e.g., blood vessel), the sealing compound may be injected through the delivery sheath into the extra-vascular space and/or puncture track, the needle preventing any substantial amount of the sealing compound from entering the body lumen by providing a temporary seal at the arteriotomy. Alternatively, a catheter or other device may be advanced over the needle and/or over a guide wire advanced through the needle into the body lumen to temporarily seal the body lumen from the puncture. Once the sealing compound is delivered into the puncture, the needle, delivery sheath, and/or catheter may be removed, leaving the guide wire in place. An introducer sheath (which may be the same or different than the tubular member used to deliver the sealing compound) may be introduced and advanced until its distal end enters the body lumen.

After delivering the sealing compound, the body lumen may be accessed via the introducer sheath to perform a medical procedure via the body lumen. For example, one or more instruments may be introduced through the introducer sheath into the body lumen to perform one or more intravascular procedures (e.g., diagnostic and/or therapeutic procedures) within the patient's vasculature.

After completing the procedure(s), the introducer sheath is withdrawn from the puncture, and the sealing compound residing in the extra-vascular tissue space proximate the body lumen and/or within the puncture site may at least partially occlude the puncture to facilitate sealing and/or hemostasis.

In an additional embodiment, for example, the delivery device for the sealing compound may be a needle through which the sealing compound may be injected into the tissue space posterior to the skin and anterior to the body lumen (e.g. blood vessel) to be accessed, such that the sealing compound at least partially fills the space and/or permeates into the intervening tissue surrounding the body lumen. Subsequently, an access needle may be inserted into the patient's skin, through the intervening tissue treated with the sealing compound, and into a blood vessel or other body lumen, followed by the insertion of the guide wire. The access needle may then be removed, leaving the guide wire in place. An introducer sheath may then be introduced and advanced over the guide wire until it enters the body lumen.

After delivering the introducer sheath, the body lumen may be accessed via the introducer sheath to perform a medical procedure via the body lumen. After completing the procedure(s), the introducer sheath may be withdrawn from the puncture, leaving the sealing compound residing in the tissue space proximate the body lumen and/or within the puncture site at least partially occluding the puncture, e.g., to facilitate sealing and/or hemostasis.

In some embodiments, the sealing compound, e.g., a lyophilized hydrogel, may be exposed to bodily fluids from the body lumen, whereupon the sealing compound may hydrate and/or expand to facilitate hemostasis within the puncture when the introducer sheath is withdrawn after the procedure. Optionally, external pressure may be applied to the intervening tissue to enhance hemostasis within the puncture and/or another sealing compound may be delivered into the puncture.

In accordance with another aspect of the invention, an apparatus is provided for sealing a puncture extending through tissue, the apparatus including an elongate tubular member having a proximal end, a distal end terminating in a distal tip sized and/or shaped for insertion into the puncture, and a lumen extending between the proximal and distal ends. A sealing compound is carried on an exterior of the tubular member proximal the distal tip such that the sealing compound is disposed within the puncture when the tubular member is introduced into the puncture. By way of example, the sealing compound may include a hydrogel.

Optionally, a cover may extend along the exterior of the tubular member such that the cover covers the sealing compound, the cover being at least partially removable to expose the sealing compound. A lubricious coating may be provided on the exterior of the tubular member, and the sealing compound may overly the lubricious coating to facilitate the tubular member being slidable, e.g., proximally, relative to the sealing compound.

In accordance with yet another aspect of the invention, a method for sealing a puncture extending through tissue is provided, wherein a tubular member, e.g., a delivery sheath and/or introducer sheath, carrying a sealing compound on an exterior thereof is introduced into the puncture. A body lumen is accessed through the puncture, e.g., through the tubular member or a separate introducer sheath, to perform a medical procedure via the body lumen. When the introducer sheath is withdrawn from the puncture, i.e., after completing the medical procedure, the sealing compound remains within the puncture to at least partially seal the puncture. For example, the sealing compound may be a lyophilized hydrogel that, when exposed to bodily fluids from the body lumen, expands to facilitate hemostasis within the puncture.

Other objects and features of the invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a perspective view of a system for sealing a puncture, including a delivery sheath, a needle, a guide wire, and a syringe assembly for delivering sealing compound.

FIGS. 2A-2D are cross-sectional views of a patient's body, illustrating exemplary methods for sealing a puncture extending between the patient's skin and a blood vessel.

FIG. 3 is a cross-sectional view of a patient's body, illustrating another exemplary method for sealing a puncture extending between the patient's skin and a blood vessel.

FIGS. 4A and 4B are cross-sectional views of a patient's body illustrating yet another exemplary method for sealing a puncture extending between the patient's skin and a blood vessel.

FIGS. 5A and 5B are cross-sectional views of a patient's body illustrating still another exemplary method for sealing a puncture extending between the patient's skin and a blood vessel.

FIG. 6 is a cross-sectional side view of another apparatus for sealing a puncture.

### DETAILED DESCRIPTION OF THE ILLUSTRATED EMBODIMENTS

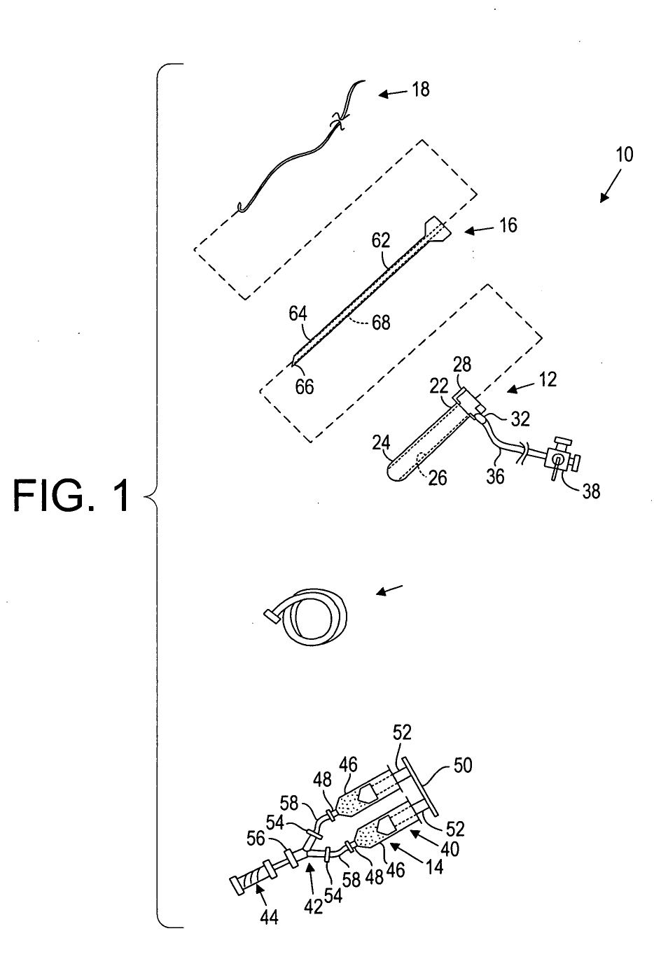

Turning to the drawings, FIG. 1 is a system 10 for sealing a puncture through tissue, e.g., a percutaneous puncture for accessing an artery or other blood vessel (not shown). Generally, the system 10 includes a delivery sheath 12 and a delivery device 14 for delivering a sealing compound into the puncture. In the illustrated embodiment, the system 10 includes other components, including a needle 16 for creating the puncture, a guide wire 18, and tubing 20. In addition or alternatively, the system 10 may include other or further components for creating the puncture, delivering the delivery sheath 12 and/or guide wire 18 into a body lumen, and/or introducing instruments into the puncture (such as a standard introducer sheath, not shown), as are known to those of skill in the art.

The delivery sheath 12 generally is an elongate tubular member including a proximal end 22, a distal end 24, and a lumen 26 extending between the proximal and distal ends 22, 24. The delivery sheath 12 terminates in a tapered distal tip 25 for facilitating advancing the delivery sheath 12 substantially atraumatically through tissue into a puncture, as is known to those skilled in the art. Alternatively, the distal end of the delivery sheath 12 may include one or more side outlet ports (not shown) to direct the sealing compound during delivery. Exemplary materials for the delivery sheath 12 may include plastics, such as polyamide, PEEK, nylon, PET, PEBAX, and polyethylene, metals, such as stainless steel, and nickel titanium, and/or composite materials.

A housing 28 may be attached to or otherwise provided on the proximal end 22 of the delivery sheath 12. The housing 28 may include one or more side ports 32 that communicate with an interior of the housing 28 and the lumen 26 of the delivery sheath 12. Preferably, at least one side port 32 is provided that includes a section of flexible tubing 36 terminating in a manual shut-off valve 38 and/or a luer lock or other connector (not shown), e.g., to facilitate connecting tubing 20 and the like to the side port 32. The housing 28 may also include one or more seals (not shown), e.g., a hemostatic seal, for sealing the lumen 26 of the delivery sheath 12, yet accommodating inserting the needle 16 and/or one or more instruments (not shown) into the lumen 26 of the delivery sheath 12 while preventing body fluids, such as blood, from escaping proximally from the delivery sheath 12, as is known in the art.

The delivery device 14 may include a single syringe, or a multiple syringe assembly. As shown in FIG. 1, the delivery device 14 is a dual syringe assembly 40 that includes two components of a sealing compound, a “Y” fitting 42, and a static mixer 44. The syringe assembly 40 includes a pair of syringe barrels 46, including outlets 48 and a plunger assembly 50 slidable into the barrels 46 to cause the components therein to be injected through the outlets 48. A pair of plungers 52 are coupled to one another and yet are received in respective barrels 46. In this manner, both plungers 52 may be manually depressed substantially simultaneously to inject the components together from the syringe barrels 46. Alternatively, a system for automatically advancing the plungers 52 and/or otherwise injecting the components in the barrels 50 may be used.

The “Y” fitting 42 includes proximal sections 54 that communicate with a single distal section 56. In this manner, the “Y” fitting 42 may be connectable to outlets 48 of-the syringe barrels 46, e.g., by tubing 58 or directly (not shown), such that the components ejected out of the barrels 46 may mix before being injected into the side port 32 of the delivery sheath 12. The proximal and distal sections 54, 56 may include connectors, e.g., luer lock connectors and the like (not shown), for connecting with the outlets 48 of the syringes 46 and/or with the mixer 44, tubing 20, 58, and/or the side port 32 of the introducer sheath assembly 12. The mixer 44 may be a tubular body including vanes or other internal structures (not shown) that enhance the components mixing thoroughly together as they pass therethrough. Similar to the “Y” fitting 42, the mixer 44 may include connectors (not shown) for releasably or substantially permanently connecting the mixer 44 to the “Y” fitting 42, tubing 20, and the like.

Respective precursor polymer components may be provided in each syringe barrel 46 of the syringe assembly 40 that, when mixed together, are activated to form a hydrogel. Additional information on such hydrogels and systems for injecting them are disclosed in U.S. Pat. Nos. 6,152,943, 6,165,201, 6,179,862, 6,514,534, and 6,379,373, and in co-pending applications Ser. No. 09/776,120 filed Feb. 2, 2001, Ser. No. 10/010,715 filed Nov. 9, 2001, Ser. No. 10/068,807 filed Feb. 5, 2002, and Ser. No. 10/454,362, filed Jun. 4, 2003. The disclosures of these references and any others cited therein are expressly incorporated by reference herein.

In the illustrated embodiment, the system 10 includes a needle 16 to facilitate inserting the delivery sheath 12 through tissue. The needle 16 may be a substantially rigid elongate tube, e.g., made from stainless steel and the like, including a proximal portion 62, a distal portion 64 terminating in a beveled or otherwise sharpened distal tip 66, and a lumen 68 extending between the proximal and distal portions 62, 64. The proximal portion 62 of the needle 16 may include one or more seals, e.g., similar to the housing 28 on the delivery sheath 12, to facilitate inserting an instrument, such as guide wire 18, through the lumen 68 while substantially sealing the needle 16 from fluid flow therethrough. The guide wire 18 may include one or more known guide wires, e.g., including a “J” tip and the like, as is well known in the art.

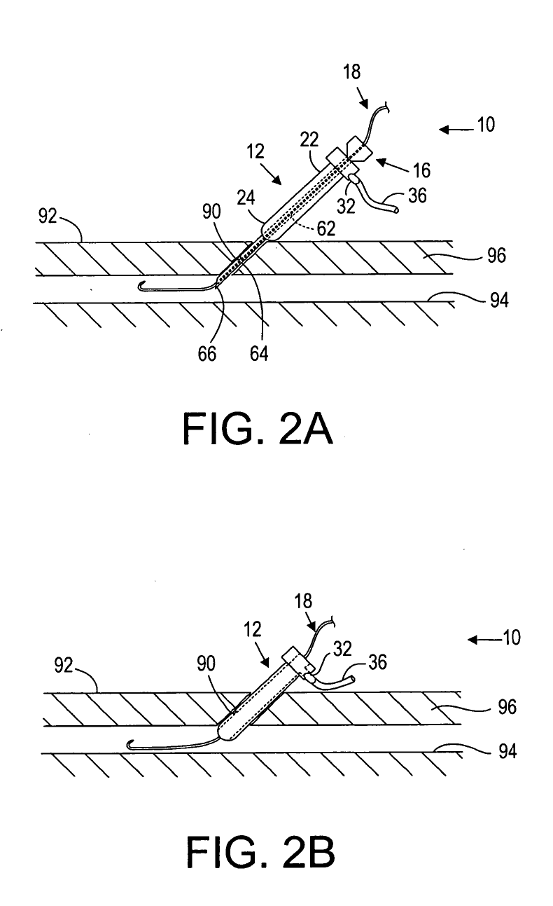

Turning to FIGS. 2A-2D, an exemplary method for sealing a passage through tissue is shown, e.g., using the system 10 of FIG. 1. In the illustrated embodiment, the passage is a percutaneous puncture 90 extending from a patient's skin 92 to a blood vessel or other body lumen 94. For example, the vessel 94 may be a peripheral artery, e.g., a femoral artery, a carotid artery, and the like. It will be appreciated that systems and methods constructed and undertaken in accordance with various embodiments of the invention may be used to seal other passages through tissue within a patient's body.

Initially, as shown in FIG. 2A, the delivery sheath 12 may be introduced into the puncture 90 such that the distal end 24 of the delivery sheath 12 is disposed within the vessel 94. For example, the delivery sheath 12 may be disposed over the proximal portion 62 of the needle 16 such that the distal end 24 of the delivery sheath 12 is located proximal to a distal portion 64 of the needle 16. The sharpened distal tip 66 of the needle 16 may be inserted into the patient's skin 92, and through any intervening tissue 96 into the vessel 94, thereby creating the puncture 90. Once the distal tip 66 is positioned within the vessel 94, the delivery sheath 12 may be advanced distally over the needle 16 into the puncture 90 until the distal tip 26 enters the vessel 94. The guide wire 18 may be advanced through the needle 16 into the vessel 94 either before or after the delivery sheath 12 is advanced into the puncture 90.

Alternatively, a hollow needle, similar to needle 16, may be inserted through a patient's skin and intervening tissue into a blood vessel without the delivery sheath 12. A guide wire, similar to guide wire 18, may be passed through a lumen of the needle into the vessel 94, whereupon the needle may be removed. The delivery sheath 12 may then be advanced over the guide wire into the vessel 94, e.g., in conjunction with or subsequent to one or more tubular dilators (not shown). It will be appreciated by those skilled in the art that the delivery sheath 12 may be introduced into the puncture 90 using other conventional methods known for introducing introducer sheaths through intervening tissue into a blood vessel.

As shown in FIG. 2B, once the delivery sheath 12 and guide wire 18 are positioned in the vessel 94, the needle 16 may be removed from the puncture 90, leaving the delivery sheath 12 and guide wire 18 in place. Then, as shown in FIG. 2C, the delivery sheath 12 may be partially withdrawn from the puncture 90 until the distal end 24 of the delivery sheath 12 is located proximal to the vessel 94, i.e., within the intervening tissue 96.

Optionally, the side port 32 may be used as a bleed back port to assist positioning the delivery sheath 12 in the puncture 90. For example, with the shut-off valve 38 open, blood may flow proximally from the vessel 94 through the delivery sheath 12 and out the side port 32. When the delivery sheath 12 is retracted, the distal end 24 may be withdrawn from the vessel 94, whereupon blood flow out the side port 32 may stop, indicating that the distal end 24 of the delivery sheath 12 is located within the puncture 90. Alternatively, visual markers (not shown) may be provided on the exterior of the delivery sheath 12 that may be used to measure or provide other visual indication that the delivery sheath 12 has been withdrawn sufficiently from the vessel 94.

A sealing compound 99 may then be delivered into the puncture 90, e.g., such that the sealing compound 99 at least partially surrounds the delivery sheath 12 and/or extends towards the vessel 94. In one embodiment, the sealing compound 99 is a liquid or other flowable material that may be injected into the puncture 90 such that the sealing compound 99 permeates into the intervening tissue 96 surrounding the puncture 90. In one embodiment, the sealing compound 99 may include one or multiple component precursor polymers that create a hydrogel when mixed together and/or upon contacting tissue fluids, as described above. Such a hydrogel sealing compound may be particularly useful, because it may be substantially harmless to the patient if it leaks into the vessel 94. Unlike collagen or other hemostasis-promoting materials, appropriately selected hydrogel precursor polymers do not cause thrombosis and/or embolism when exposed to blood. In fact, such precursor polymers, if exposed within a vessel, will simply dilute and flow away, where they may be safely metabolized naturally without substantial risk of creating thrombus.

In one embodiment, a two-part sealing compound is delivered into the puncture 90 using a dual syringe assembly 40, similar to that shown in FIG. 1 and described above. The precursor polymers or other components in the syringe barrels 46 may be mixed or otherwise prepared before the procedure using known methods. For example, the “Y” fitting 42, mixer 44, and/or tubing 20, 58 may be coupled to one another and/or to the outlets 48 before the procedure or at the time of injection. Similarly, tubing 20 may be connected to the side port 32 before the procedure or immediately before injection. Preferably, the tubing 20 is connected to the side port 32 immediately before the injection so that the tubing 20 does not obstruct or otherwise interfere with introducing the delivery sheath 12, needle 16, and/or guide wire 18, as described above.

Once the delivery sheath 12 is coupled to the deliver device 14, the plunger assembly 50 may be manually (or optionally automatically, upon actuation) depressed, advancing the plungers 52 substantially simultaneously into the barrels 46, and delivering the precursor polymers substantially simultaneously from the outlets 48. The precursor polymers mix in the “Y“fitting 42 and mixer 44 into a liquid sealing compound, and are then delivered into the side port 32 of the delivery sheath 12 via tubing 20. The liquid sealing compound 99 exits the distal end 24 of the delivery sheath 12, and enters the puncture 90, where it at least partially surrounds the delivery sheath 12 and/or permeates into the intervening tissue 96.

The sealing compound 99 should be permitted sufficient time to “gel” or cure and/or solidify within the puncture 90, e.g., between about five (5) and one hundred eighty (180) seconds. Once the sealing compound is delivered into the puncture 90 and/or at least partially gelled, the delivery sheath 12 is removed while the vessel 94 is compressed proximally (upstream relative to the vessel 94) to prevent blood from leaking out of the puncture 90. An introducer sheath (not shown), such as those known in the art, may be introduced and advanced over the guide wire 18 until the distal end 24 enters the vessel 94, whereupon the compression is relieved to allow blood flow to resume in the vessel 94.

In further alternatives, the delivery sheath 12 may include one or more secondary lumens (not shown) located in the wall of the delivery sheath 12 that extend from the proximal end 22 to an intermediate location proximal to the distal end 24. One or more side outlets (also not shown) may be provided in the side wall of the delivery sheath 12 and one or more inlet side ports (also not shown) may be provided in the housing 28 that communicate with the secondary lumen(s). For example, if a single secondary lumen is provided, the tubing from the delivery device may be coupled to the inlet side port for delivering the sealing compound via the secondary lumen to the side wall outlet. Alternatively, two secondary lumens may be provided, and each precursor polymer may be delivered into a respective secondary lumen such that precursor polymers mix together when they exit the side outlets within the puncture 90.

One advantage of these alternatives is that the sealing compound may be delivered into the intervening tissue surrounding the puncture 90 without having to retract the delivery sheath 12, thereby reducing handling of the delivery sheath 12. In addition, these alternatives may allow the lumen 28 to remain unobstructed, since the secondary lumen(s) is(are) used to deliver the sealing compound, which may gel or otherwise solidify to obstruct the secondary lumen(s). With the lumen 28 unobstructed by sealing compound, the delivery sheath 12 may be used as an introducer sheath subsequent to delivering the sealing compound, as explained further below.

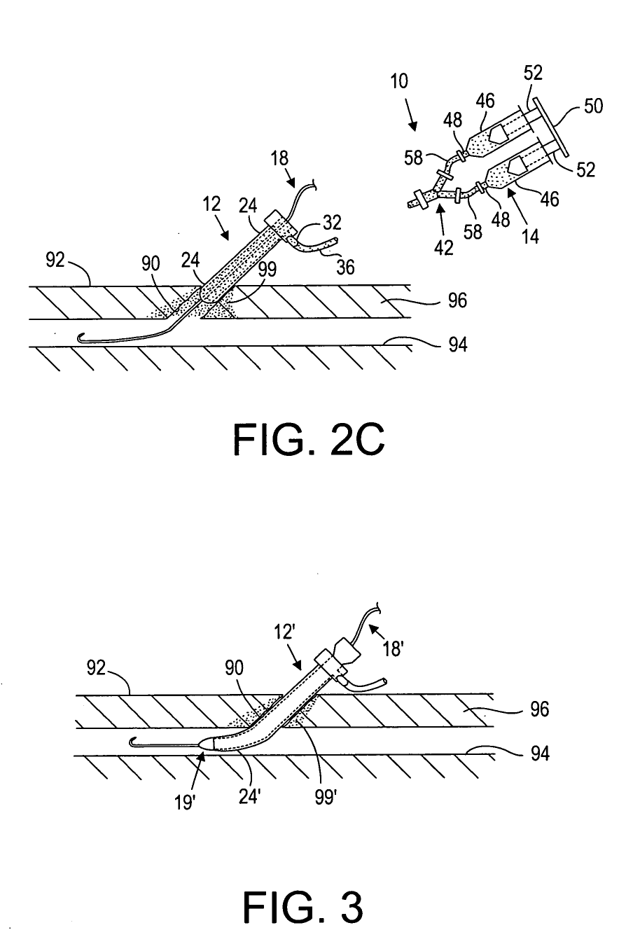

Turning to FIG. 3,a delivery sheath 12,′ including one or more secondary lumens (not shown), is advanced over a needle (not shown), similar to the needle 16 of FIG. 1 until the distal end 24′ enters the puncture 90 but does not enter the vessel 94. The needle 16′ may then be removed, and sealing compound 99′ delivered into the puncture 90 through the one or more secondary lumens. The delivery sheath 12′ may then be advanced over the guide wire 18′ until the distal end 24′ is disposed within the vessel 94, e.g., in conjunction with one or more dilators (an exemplary dilator 19′ being shown in FIG. 3), as is known to those skilled in the art. Alternatively, the needle may remain in the puncture 90 while the sealing compound 99′ is delivered, and removed before or after the delivery sheath 12′ is advanced into the vessel 94. In still another alternative, the delivery sheath 12′ may be removed after delivering the sealing compound 99,′ leaving the guide wire 18′ in place. An introducer sheath (not shown), in conjunction with one or more dilators (also not shown), may be advanced into the puncture 90 until the distal end of the introducer sheath enters the vessel 94.

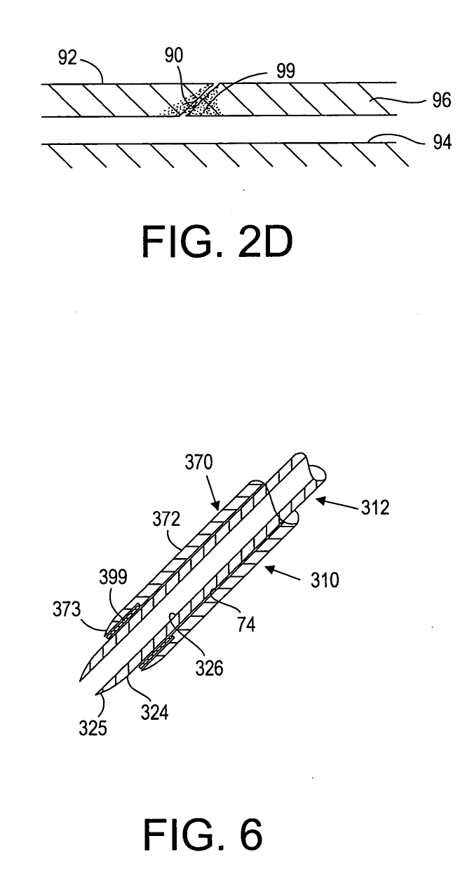

Turning to FIGS. 4A and 4B, another method for pre-sealing a puncture 90 is shown. Similar to the methods described above, a needle 116 may be inserted into tissue 96 to create puncture 90 and advanced until distal tip 166 enters vessel 94, as shown in FIG. 4A. Delivery sheath 112 (which may be similar to any of the embodiments described herein) may be advanced over the needle 116 until distal end 124 of the delivery sheath 112 is disposed proximal to the vessel 94. For example, the delivery sheath 112 may be disposed initially on a proximal portion of the needle 116 when the needle 116 is inserted (not shown), may be advanced over the needle 116 after the puncture 90 is created, or may be advanced together with the needle 116, similar to the embodiments described above.

As shown in FIG. 4B, sealing compound 199 may then be delivered through lumen 126 of the delivery sheath 112 into the puncture 99. In this embodiment, the needle 116 remains in the puncture 90 such that the distal tip 166 extends into the vessel 94, e.g., to at least partially and preferably substantially seal the vessel 94 from the puncture 90. In this manner, the sealing compound 99 may be introduced into the puncture 90 via the delivery sheath 112, with the needle 116 preventing substantial amounts of the sealing compound 99 from entering the vessel 94. This alternative may allow thrombogenic and/or bioabsorbable sealing materials, such as collagen thrombin, fibrin, polyglycolic acids (PGA's), polyactides (PLA's), natural or synthetic tissue adhesives, and the like, to be introduced into the puncture 90 without substantial risk of their entering the vessel 94. Optionally, a catheter or other device (not shown) may be advanced over the needle 116 to further seal the vessel 94 from the puncture 90 before the sealing compound 99 is introduced.

A guide wire.118 may be advanced through the needle 116 before or after delivering the sealing compound 99. After the sealing compound 99 is delivered, the needle 116, delivery sheath 112, and/or catheter may be removed before an introducer sheath (not shown) is introduced into the puncture 90 over the guide wire 118 and advanced into the vessel 94. Alternatively, the delivery sheath 112 may be advanced over the guide wire until the distal end 124 enters the vessel 94, and used as an introducer sheath, e.g., if the delivery sheath includes one or more secondary lumens through which the sealing compound 99 is. delivered. Thus, the sealing compound 99 may “pre-seal” the puncture 90, e.g., bulking the puncture 90 and/or creating a pillowing effect that may enhance sealing of the puncture 90, as described further below.

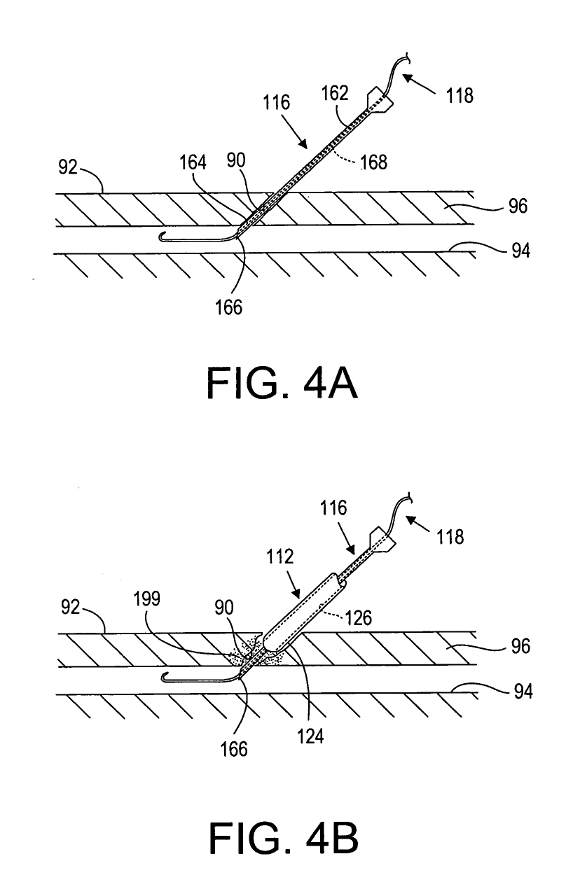

Turning to FIG. 5A and 5B, another method is shown for pre-sealing a puncture 90 through tissue 96, e.g., communicating with vessel 94 or other body lumen. As shown in FIG. 5A, a needle 116, including a sharpened distal tip 266 and a lumen 268, is inserted through the patient's skin 92 into tissue 96 without penetrating into the vessel 94. The needle 116 may include a side port 269 that may be coupled to a delivery device, such as the dual syringe assembly 14 shown in FIG. 1 or other source of sealing compound (not shown in FIG. 5A). Alternatively, a delivery device (not shown) may be inserted into the lumen 268, e.g., through one or more seals (also not shown) at the proximal end 262 of the needle 216.

Sealing compound (such as any of those described herein) may be delivered into the side port 269, through the lumen 268, and out the distal tip 266 of the needle 216 into the extra-vascular space above the vessel 94. Because the wall of the vessel 94 has not been pierced, the sealing compound 299 may fill the puncture 90 and/or permeate into the surrounding tissue 96, thereby bulking the puncture 90 and/or creating a pillowing effect above the vessel 94.

After delivering the sealing compound 299, the needle 216 may be removed, and another needle 216′ (which may be similar to the needle 216) advanced through the tissue 96 and/or through the sealing compound 299 until its distal tip 266′ penetrates the wall of the vessel 94. A guide wire 218 may be advanced through the needle 216′ into the vessel 94, the needle 216′ may be removed from the puncture 90, and an introducer sheath (not shown) may be advanced over the guide wire 218 into the vessel 94. Thus, in this embodiment, there may be no need for a separate delivery sheath or other device to deliver the sealing compound 299. Alternatively, a single needle may be used to deliver the sealing compound 299 and access the vessel 94, e.g., if the needle includes separate lumens for delivering the sealing compound 299 and advancing instruments into the vessel 94, similar to embodiments of the delivery sheath described above.

Once sealing compound is delivered into a puncture to pre-seal the puncture, e.g., using any of the methods described herein, an introducer sheath (which may be the same or different than the delivery sheaths described above) may then be used to access the vessel, e.g., to perform one or more therapeutic and/or diagnostic procedures within the patient's body. For example, one or more instruments, (not shown), may be advanced through the delivery sheath 12 and into the vessel 94, alone or in conjunction with one another, as is known in the art. The one or more instruments may include catheters, e.g., balloon catheters, stent delivery catheters, imaging catheters, and the like; guide wires; filters; electrophysiology therapy and/or mapping devices; and the like. Thus, the procedures may include one or more of stent delivery, angioplasty, atherectomy, thrombectomy, angiography, cardiac mapping, ablation, and the like.

Upon completing the procedure(s), the instruments are removed from the introducer sheath, and the introducer sheath may be withdrawn at least partially, and preferably completely, from the puncture. Turning to FIG. 2D, once the introducer sheath is withdrawn, the sealing compound 99 (which may correspond to any of the embodiments described herein) surrounding the puncture 90 facilitates sealing and/or hemostasis within the puncture 90. For example, if the sealing compound 99 has gelled and/or solidified in the puncture 90 around the introducer sheath (not shown), the sealing compound 99 may be at least partially compressed between the introducer sheath and the surrounding tissue 96. In addition or alternatively, the vessel 94 proximal to the puncture site may be compressed to cease the blood flow before or while removing the introducer sheath.

When the introducer sheath is withdrawn from the puncture 90, the sealing compound 99 may expand inwardly into the puncture 90, thereby facilitating sealing and/or hemostasis. In addition or alternatively, the intervening tissue 96 surrounding the puncture 90 may at least partially recoil, further directing the sealing compound 99 into the puncture 90 to at least partially seal the puncture 90. If the sealing compound 99 is a lyophilized hydrogel or other material, e.g., in solid or powder form, the sealing compound 99 may be exposed to fluid, e.g., blood, from the vessel 94 after the introducer sheath is removed, causing the sealing compound 99 to hydrate further and/or swell, thereby further enhancing sealing the puncture 90.

Thus, as shown in FIG. 2D, sealing compound 99 may enhance hemostasis, thereby preventing substantial blood from escaping from puncture 90. Optionally, thereafter, external manual pressure may be applied to the skin 92 overlying the puncture 90 until complete hemostasis occurs. Preloading the sealing compound 99,in the puncture 90 may substantially reduce the time for hemostasis to occur, as compared to external pressure alone.

In alternative embodiments, one or more other sealing devices (not shown) may be introduced into the puncture 90 after the above-identified procedure(s) to further enhance hemostasis and/or sealing the puncture 90. For example, additional liquid sealing compound, e.g., hydrogel and/or hydrogel prepolymers, may be injected into the puncture 90 using the delivery sheath 12 or other devices. Exemplary apparatus and methods for sealing a puncture after a procedure are disclosed in above-incorporated application Ser. No. 10/454,362. Alternatively, other known sealing materials, e.g., plugs, clips, and the like, may be delivered into the puncture 90 after the procedure(s). Such sealing materials may include those that mechanically close a puncture, e.g., sutures, anchors, clips, those that promote clotting, e.g., thrombin, collagen, fibrin etc., and/or those that adhere, e.g.,cyanoacrylates, fibrin glue, protein-based adhesives, synthetic adhesives, synthetic sealants, and the like. Thus, the preloaded sealing compound may be used to enhance sealing in addition to, or instead of, other known devices and methods.

In yet another alternative, a sealing compound may be introduced into the puncture 90 during the procedure, e.g., at any time after creation of the puncture 90 and/or before completion of the procedure. For example, an introducer sheath or other supplementary tubular member (not shown) may be advanced into the puncture 90 to at least partially fill the puncture 90 with sealing compound before removal of the final instruments, delivery sheath, and/or guide wire.

In addition to liquid hydrogel and/or precursor polymers, other sealing compounds may be delivered into the puncture 90 before accessing the vessel 94 to perform one or more procedures. For example, a solid hydrogel plug or powder may be delivered into the puncture 90, e.g., via the delivery sheath 12, shown in FIG. 1. Alternatively, other sealing materials, such as collagen or other hemostasis-promoting materials may be delivered into the puncture 90, as long as care is taken not to expose any thrombogenic materials within the vessel 94.

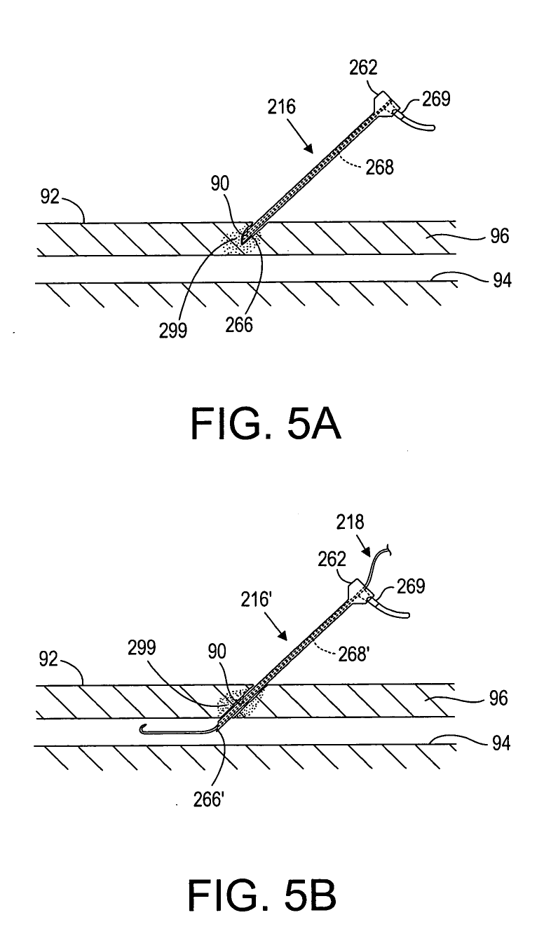

Turning to FIG. 6., an apparatus 310 is shown for delivering a plug of sealing material into a puncture (not shown) before accessing a vessel (also not shown) via the puncture to perform one or more medical procedures. Generally, the apparatus 310 includes a delivery sheath 312, which may be identical or similar to the delivery sheath 12 shown in FIG. 1 and described above. In addition, a sealing compound 399 is carried on an exterior of the delivery sheath 312 proximal to its distal tip 325.

The sealing compound 399 is an annular plug or other mass of lyophilized hydrogel, such as that disclosed in U.S. Pat. No. 6,605,294. The disclosures of this reference and any others cited therein are expressly incorporated herein by reference. The sealing compound 399 may be in a powder form, a hollow tube, or may be a solid mass, or rod. The sealing compound 399 may have a pre-delivery (e.g., pre-expanded or pre-swell state) diameter between about one and twenty five millimeters (1-25 mm), preferably between about five and ten millimeters (5-10 mm), and/or a length of between about five and twenty five millimeters (5-25 mm), preferably between about five and ten millimeters (5-10 mm). It will be appreciated by those skilled in the art that other shapes and/or configurations may be provided for the sealing compound 399.

Alternatively, other materials may be carried on the exterior of the delivery sheath 312 instead of a hydrogel, e.g., one or more biocompatible materials, such as collagen, thrombin, fibrin, polyglycolic acids (PGA's), polyactides (PLA's), and the like, which may be at least partially absorbed by the body over time.

Optionally, a cover 370 may be provided over the delivery sheath 312 that may at least partially cover the sealing compound 399. The sealing compound 399 may be pre-mounted to the delivery sheath 310 in its original pre-swelled size and/or squeezed or compressed into a smaller size/dimension in order to reduce its profile. The cover 370 is a relatively thin-walled sheath or peel-away skin, including a tapered distal tip 373 to facilitate atraumatic advancement through tissue. The cover 370 may be slidable relative to the delivery sheath 312, e.g., such that the cover 370 may be retracted to expose the sealing compound 199. Alternatively, the cover 370 may include one or more weakened regions (not shown) that may be separate when the cover 370 pulled proximally or apart to allow the cover 370 to be removed entirely from around the delivery sheath 312.

During use, the apparatus 310 may be introduced into a puncture (not shown), similar to the systems and methods described above for introducing the delivery sheath 12 shown in FIG. 1. The delivery sheath 312 may be introduced into the puncture, e.g., using a needle, guidewire, and/or other devices (not shown), as described above. With the distal end 324 of the delivery sheath 312 disposed within the vessel, the sealing compound 399 may be deposited within the puncture by moving the cover 370 to expose the sealing compound 399, e.g., by slidably retracting the cover 370 partially, or removing the cover 370 completely.

With the cover 370 retracted or removed, the sealing compound 399 is exposed within the puncture and/or to any fluid located within the puncture and/or surrounding tissue. For example, some fluid may be present naturally within the surrounding tissue that may at least partially hydrate the sealing compound, if the sealing compound is a lyophilized hydrogel. This would cause the sealing compound 399 to swell and/or expand within the puncture. Since the delivery sheath 312 is present, the sealing compound 399 may expand in size from the delivery sheath 312, thereby compressing the surrounding tissue.

A guide wire (not shown) may be introduced into the delivery sheath 310 (and/or through a needle used to create the puncture 90, not shown) to maintain access the body lumen 94. The delivery sheath 312 may then be removed from the puncture, and an introducer sheath (not shown) may be advanced into the puncture, similar to the embodiments described above, to perform one or more medical procedures. Alternatively, the delivery sheath 312 may be used as an introducer sheath, similar to embodiments described above. Once the medical procedure(s) is(are) performed, the introducer sheath (or delivery sheath 312) is removed from the puncture, leaving the sealing compound 399 behind. Optionally, the delivery sheath 312 may include a Teflon or other lubricious coating (not shown) on an exterior of the delivery sheath 312. The sealing compound 399 may be disposed over the coating such that the delivery-sheath 312 may be slid relative to the sealing compound 399.

If the sealing compound 399 has not fully hydrated and/or expanded, it may expand further inwardly, thereby at least partially occluding the puncture. Thus, the sealing compound 399 may expand to many times, e.g., twice, three-times, or more, its pre-swelled diameter. If the sealing compound is fully hydrated, it may expand inwardly into the puncture or the surrounding tissue may recoil to further enhance sealing and/or hemostasis of the puncture, similar to the embodiments described above. If desired, external pressure may be applied and/or another sealing device may be delivered into the puncture, also similar to the embodiments described above.

While the invention is susceptible to various modifications, and alternative forms, specific examples thereof have been shown in the drawings and are herein described in detail. It should be understood, however, that the invention is not to be limited to the particular embodiments or methods disclosed, but to the contrary, the invention is to cover all modifications, equivalents and alternatives falling within the scope of the appended claims.

1. A method for sealing a puncture extending through intervening tissue to a body lumen, the method comprising:
introducing a delivery sheath into the puncture;
introducing hydrogel components into the puncture to create a hydrogel within the puncture;
after introducing the hydrogel components into the puncture, introducing one or more instruments through the puncture via an introducer sheath to perform a medical procedure via the body lumen; and
withdrawing the introducer sheath from the puncture after completing the medical procedure such that the sealing compound is exposed to bodily fluids from the body lumen, whereupon the sealing compound expands to at least partially seal the puncture and facilitate hemostasis within the puncture.

2. A method for sealing a puncture extending through tissue, comprising:
introducing a tubular member into the puncture;
introducing a sealing compound into the puncture;
after introducing the sealing compound, accessing the body lumen through the puncture to perform a medical procedure via the body lumen.

3. The method of claim 2, wherein the sealing compound comprises a hydrogel.

4. The method of claim 2, wherein the sealing compound comprises a liquid.

5. The method of claim 2, wherein the sealing compound is delivered from one or more syringes into the tubular member.

6. The method of claim 2, wherein the sealing compound is a hydrogel formed by precursor polymer components.

7. The method of claim 2, wherein the sealing compound is introduced by:
positioning the tubular member such that a distal end of the tubular member is disposed within intervening tissue surrounding the puncture; and
injecting the sealing compound through the tubular member such that the sealing compound exits the distal end and permeates the intervening tissue.

8. The method of claim 2, wherein the tubular member is introduced by:
inserting a sharpened elongate member through the intervening tissue and into the body lumen to create the puncture; and
advancing the tubular member over the sharpened elongate member and into the puncture.

9. The method of claim 8, wherein the tubular member is positioned over a proximal portion of the sharpened elongate member as the sharpened elongate member is inserted through the intervening tissue.

10. The method of claim 8, further comprising removing the sharpened elongate member from the tubular member with a distal end of the tubular member extending into the body lumen.

11. The method of claim 2, wherein the body lumen is accessed by introducing one or more instruments through the tubular member into the body lumen.

12. The method of claim 2, further comprising withdrawing the tubular member from the puncture after completing the medical procedure via the body lumen.

13. The method of claim 12, wherein the sealing compound is exposed to bodily fluids from the body lumen when the tubular member is withdrawn, whereupon the sealing compound expands to facilitate hemostasis within the puncture.

14. The method of claim 12, further comprising applying external pressure to tissue proximate the puncture to enhance hemostasis within the puncture.

15. The method of claim 12, further comprising introducing one or more further sealing compounds into the puncture after performing the medical procedure.

16. The method of claim 2, wherein the sealing compound is disposed around the tubular member with a cover overlying the sealing compound, and wherein the sealing compound is introduced into the puncture by at least partially removing the cover to expose the sealing compound.

17. An apparatus for sealing a puncture extending through tissue, comprising:
an elongate tubular member comprising a proximal end, a distal end terminating in a distal tip, the distal tip sized and shaped for insertion into the puncture, the tubular member further comprising a lumen extending between the proximal and distal ends; and
a sealing compound carried on an exterior of the tubular member proximal the distal tip such that the sealing compound may be deposited within the puncture when the tubular member is introduced therein.

18. The apparatus of claim 17, further comprising a cover extending along the exterior of the tubular member and covering the sealing compound, the cover being selectively movable to expose the sealing compound.

19. The apparatus of claim 18, further comprising a lubricious coating on the exterior of the tubular member and underlying the sealing compound to allow the tubular member to slide relative to the sealing compound.

20. A method for sealing a puncture extending through tissue, comprising:
introducing a tubular member into the puncture, the tubular member carrying a sealing compound on an exterior thereof;
accessing the body lumen through the tubular member to perform a medical procedure via the body lumen; and
withdrawing the tubular member from the puncture after completing the medical procedure such that the sealing compound remains within the puncture to at least partially seal the puncture.

21. The method of claim 20, wherein the sealing compound is exposed to bodily fluids from the body lumen upon withdrawing the tubular member, whereupon the sealing compound expands to facilitate hemostasis within the puncture.

22. The method of claim 21, further comprising applying external pressure to tissue proximate the puncture to enhance hemostasis within the puncture.

23. The method of claim 21, wherein a cover overlies the sealing compound, the method further comprising at least partially removing the cover overlying the sealing compound to expose the sealing compound within the puncture.

24. A method for sealing a puncture extending through tissue, comprising:
introducing a sealing compound into the puncture to at least partially fill the puncture;
after introducing the sealing compound, accessing the body lumen through the puncture to perform a medical procedure via the body lumen.

25. The method of claim 24, the sealing compound comprising a liquid, wherein the sealing compound is introduced into the puncture by introducing a tubular member into the puncture and injecting the sealing compound through the tubular member into the puncture.

26. The method of claim 25, wherein introducing the sealing compound into the puncture further comprises:
positioning the tubular member such that a distal end of the tubular member is disposed within intervening tissue surrounding the puncture; and
injecting the sealing compound through the distal end of the tubular member such that the sealing compound at least partially surrounds the tubular member.

27. The method of claim 26, the tubular-member comprising a first needle, wherein the tubular member is positioned by inserting the first needle into the intervening tissue without penetrating a wall of the body lumen.

28. The method of claim 27, further comprising advancing the first needle distally to penetrate the wall of the body lumen after injecting the sealing compound.

29. The method of claim 27, further comprising:
removing the first needle from the puncture;
inserting a second needle into the intervening tissue to penetrate the wall of the body lumen; and
advancing a guide wire through the second needle into the body lumen.

30. The method of claim 29, further comprising advancing an introducer sheath over the guide wire to access the body lumen to perform the medical procedure.

31. The method of claim 24, wherein the body lumen is accessed by introducing one or more instruments through the tubular member into the body lumen.

32. The method of claim 31, further comprising withdrawing the tubular member from the puncture after completing the medical procedure such that the sealing compound is exposed to bodily fluids from the body lumen, whereupon the sealing compound expands to facilitate hemostasis within the puncture.

33. The method of claim 24, wherein the sealing member is introduced by:
inserting a sharpened elongate member through the intervening tissue and into a body lumen to create the puncture; and
introducing the sealing compound around the sharpened elongate member.

34. The method of claim 33, wherein the sharpened elongate member at least partially seals the body lumen from the puncture to prevent the sealing compound from entering substantially into the body lumen.

35. The method of claim-33, further comprising advancing a device over the sharpened elongate member to at least partially seal the body lumen from the puncture to prevent the sealing compound from entering substantially into the body lumen.

36. The method of claim 33, further comprising withdrawing the sharpened elongate member from the puncture after introducing the sealing compound into the puncture.

37. The method of claim 33, further comprising advancing a guide wire through the sharpened elongate member into the body lumen.

38. The method of claim 37, further comprising advancing an introducer sheath over the guide wire until a distal end of the introducer sheath is disposed within the body lumen, and wherein the medical procedure is performed by introducing one or more instruments through the introducer sheath and into the body lumen.

39. The method of claim 28, further comprising applying external pressure to tissue proximate the puncture after performing the medical procedure in order to enhance hemostasis within the puncture.

40. The method of claim 28, wherein one or more instruments are introduced through the puncture into a body lumen before the sealing compound is introduced into the puncture, and wherein the medical procedure performed after introducing the sealing compound into the puncture comprises introducing one or more instruments through the puncture into the body lumen.
